(19) United States
(12) Patent Application Publication
(10) Pub. No.: US 2007/0066990 A1
(43) Pub. Date: Mar. 22, 2007

(54) Device for forming a fluid tight seal during a procedure within a hollow organ

(76) Inventors: Andrew Marsella, Boston, MA (US); Aidan Petrie, Jamestown, RI (US); Todd Sloan, Medway, MA (US); Jon T. McIntyre, Newton, MA (US); David Robson, Riverside, RI (US); Joseph M. Gordon, Mansfield, MA (US); Jeff P. Cheek, Hopkinton, MA (US); John F. Howard, Salem, MA (US)

Correspondence Address: FAY KAPLUN & MARCIN, LLP, 15O BROADWAY, SUITE 702, NEW YORK, NY, 10038, US

(21) Appl. No.: 11/230,147

(22) Filed: Sep. 19, 2005

(51) Int. Cl. A61M 29/00

(52) U.S. Cl. 606/193

(57) ABSTRACT

A device for accessing a hollow organ comprises an elongated body sized for insertion into an opening into a hollow organ, the elongated body including a working channel extending therethrough from a proximal opening which, when the seal is in an operative position, is located proximally of the opening to a distal opening which, when the seal is in the operative position, opens into the hollow organ and a seal extending around a portion of the elongated body between the proximal and distal openings, the seal expanding from an insertion configuration to a sealing configuration.

### BACKGROUND

The treatment of tissue masses (e.g., fibroids and tumors) often involves the destruction of tissue. For example, local ablation of a tissue mass may be carried out by inserting a therapeutic device thereinto to destroy targeted cells. Electrical energy may be applied to the tissue mass via one or more electrodes inserted into the tissue mass or fluids with appropriate properties may be injected into the vicinity of the tissue mass to chemically necrose selected portions of the tissue mass.

A number of diagnostic and therapeutic procedures requiring access to the uterus often involve dilation of the cervix to facilitate the introduction instruments therethrough into the uterus. However, when the cervix is over dilated or patulated or, when a device accessing the uterus is manipulated during a procedure, fluid or gas may leak from the cervix. The ablation of uterine tissue may involve the application of intracavity pressure while introducing fluids into the uterus. For example, this may be done when using the Hydro Therm Ablator (HTA®) uterine endometrial ablation system or similar uterine ablation systems, when using an RF uterine ablation system or when utilizing an alternate method of treating the endometrial lining. Injuries may occur during procedures involving the application of heated fluids (e.g., to ablate the endometrium) if these fluids escape from the uterus.

Although the cervical muscle is strong and effectively creates a seal at the opening of the uterus, procedures such as these may require mechanically enhancing the seal of the cervix to prevent fluid leakage therefrom. This may also be required where the cervix is over dilated, weak or subject to significant stress from movement of a device within the cervical os.

Currently, the cervix most often seals itself, but can be enhanced by compressing the cervix with a tenaculum clamped externally therearound. Conventional tenaculums include scissor-like clamps that generate significant compression around the cervix. However, multiple clamps may be required to effectively seal the cervix around its entire circumference and placing these tenacula requires more time which may still not result in an adequate seal. Clamps placed externally to the cervix may also increase trauma and patient discomfort.

### SUMMARY OF THE INVENTION

The present invention is directed to a device for accessing a hollow organ comprising an elongated body sized for insertion into an opening into a hollow organ, the elongated body including a working channel extending therethrough from a proximal opening which, when the seal is in an operative position, is located proximally of the opening to a distal opening which, when the seal is in the operative position, opens into the hollow organ and a seal extending around a portion of the elongated body between the proximal and distal openings, the seal expanding from an insertion configuration to a sealing configuration.

### BRIEF DESCRIPTION OF DRAWINGS

FIG. 1 is a diagram showing a first embodiment of a sealing device according to the invention;

FIG. 2 is a diagram showing a second embodiment of a sealing device according to the invention;

FIG. 3 is a side elevation view showing operation of the embodiment of the sealing device shown in FIG. 2;

FIG. 4 is a diagram showing a third embodiment of a sealing device according to the invention;

FIG. 5 is a side elevation view showing operation of the embodiment of the sealing device shown in FIG. 4;

FIG. 6 is a diagram showing another embodiment of a sealing device according to the invention, including an inflatable balloon; and

FIG. 7 is a diagram showing another embodiment of a sealing device according to the invention, including two inflatable balloons.

### DETAILED DESCRIPTION

The present invention may be further understood with reference to the following description and the appended drawings, wherein like elements are referred to with the same reference numerals. The present invention is related to medical devices used to access hollow organs (e.g., the uterus) for medical treatment. In particular, the present invention relates to devices for reinforcing the fluid tight seal formed by an opening to the hollow organ (e.g., the cervix) around a medical instrument introduced into the organ therethrough.

Endometrial ablation procedures are often performed to treat menhorrhagia, or excessive uterine bleeding. As described above, ablation may be done using radio frequency (RF) methods, microwave heating or through the application of heated liquids. The heated liquids are either free flowing within the uterus or contained within a balloon or other constraining device. The Hydro Therm Ablator (HTA) circulates heated saline solution within the uterine cavity via a probe or sheath inserted into the uterus through the cervical canal. As described above, it is imperative that a fluid tight seal between the cervical canal and the HTA probe be maintained to prevent the escape of the heated fluid therefrom.

There are a wide variety of procedures carried out wherein it is desirable to maintain a fluid tight seal around an instrument inserted into a hollow organ (e.g., in the gastrointestinal tract). Such a seal may be useful in any case where a seal provided by a natural sphincter, restriction or passage into the cavity is not sufficient to provide a fluid-tight seal when a therapeutic device is inserted therethrough. A fluid tight seal may also be useful for bladder treatments. Thus, although the following description is principally directed to a cervical sealing device, those of skill in the art will understand that such description is illustrative only and that embodiments of the device may be used in procedures performed on a variety of other organs.

A device according to an exemplary embodiment of the present invention includes a seal comprising an elongated body adapted for insertion through the opening of the hollow organ (e.g., the cervix) and a plurality of elements connected to the elongated body to maintain a fluid-tight seal of the passage. The seal may also include a working channel extending therethrough, to permit a therapeutic instrument to be inserted into the hollow organ therethrough.

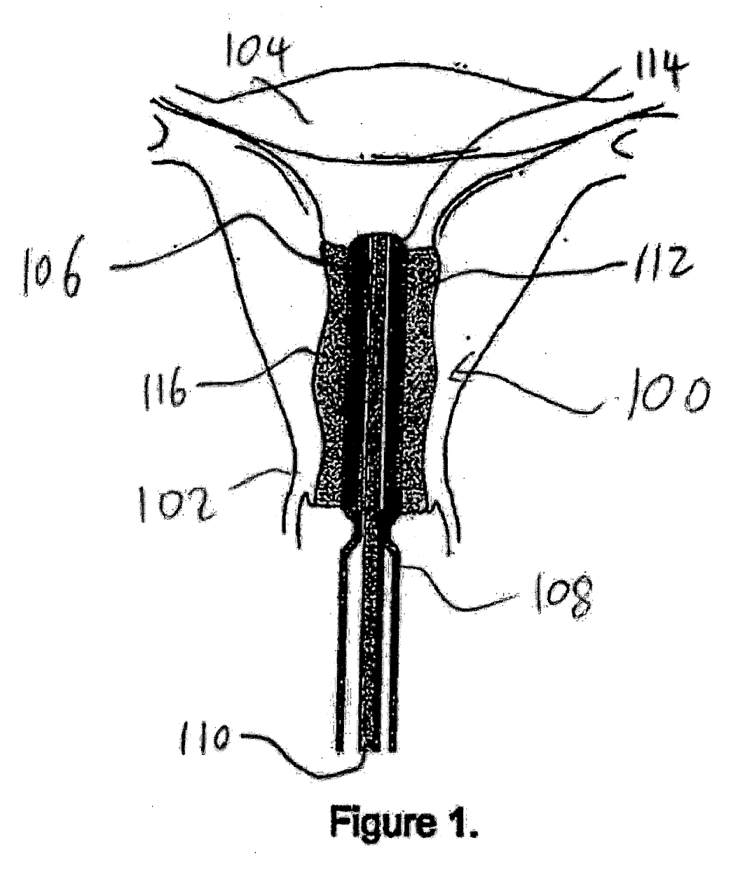

FIG. 1 shows an exemplary embodiment of a cervical seal according to the invention. The cervical seal 100 is sized, shaped and otherwise adapted for trans-cervical placement within the cervix 102, at the opening of the uterus 104. The cervical seal 100 comprises an elongated body 106, a distal end 114 of which is insertable into the uterus 104 through the cervix 102. A proximal part of the elongated body 106 serves as a control portion to manipulate the cervical seal 100. A high expansion foam element 112 is placed along a portion of the length of the elongated body 106, particularly near the distal end 114. The foam element 112 preferably extends along the length of the cervical canal so that, if a leak were to develop between the seal 100 and the walls of the cervix 102, the foam element 112 would be hydrated by the leaking fluid which would be absorbed therein. In addition, as hydration of the foam element 112 increases, the volume of the foam element 112 also increases, tightening the mechanical seal with the walls of the cervix 102.

In one embodiment, the foam element 112 includes a protective skin 116 formed of a more resilient and non-absorbent material. For example, the skin layer 116 may form a sheath placed over the entire length of the foam element 112 or a selected portion thereof during insertion into the body. Then, after the seal 100 has been properly positioned within the cervix 102, the skin layer 116 is withdrawn to expose the surface of the foam layer 116. Thus, the skin layer 116 helps to prevent hydration of the foam element 112 before reaching the desired location. In one exemplary embodiment, the foam element 112 is formed of a PVA foam. However, those skilled in the art will understand that any of a wide variety of bio-compatible, fluid-absorbing materials may be used for the foam element 112. The material of the foam element 112 is preferably selected to have mechanical properties enabling it to return to an original shape and dimension after being compressed, for example during insertion through the cervix 102.

In a similar embodiment, the foam element 112 is formed of an expanding weave of material adding a mechanically interlocking function to the material, as the tissue of the cervix 102 works its way into interstices of the weave. This exemplary embodiment of the invention more securely seats the seal 100 within the cervix 102 during the procedure.

A shaft 108 extending from the elongated body 106 of the seal 100 is used to manipulate and position the elongated body 106 and its associated elements within the cervix 102. A working channel 110 extends longitudinally within the shaft 108 and the elongated body 106 between a proximal opening 111 which, when the seal 100 is in position, remains outside the body to a distal opening 113 which opens to the uterus 104. The working channel 110 preferably extends substantially along an axis of the seal 100, to provide a passage into the uterus 104. For example, medical instruments and therapeutic devices may be inserted into the uterus 104 through the working channel 110 while the cervix remains sealed around the seal 100. In a specific exemplary embodiment, devices for carrying out endometrial ablation (e.g., using heated saline solution or other ablating fluids) may be inserted though the working channel 110 into the uterus 104.

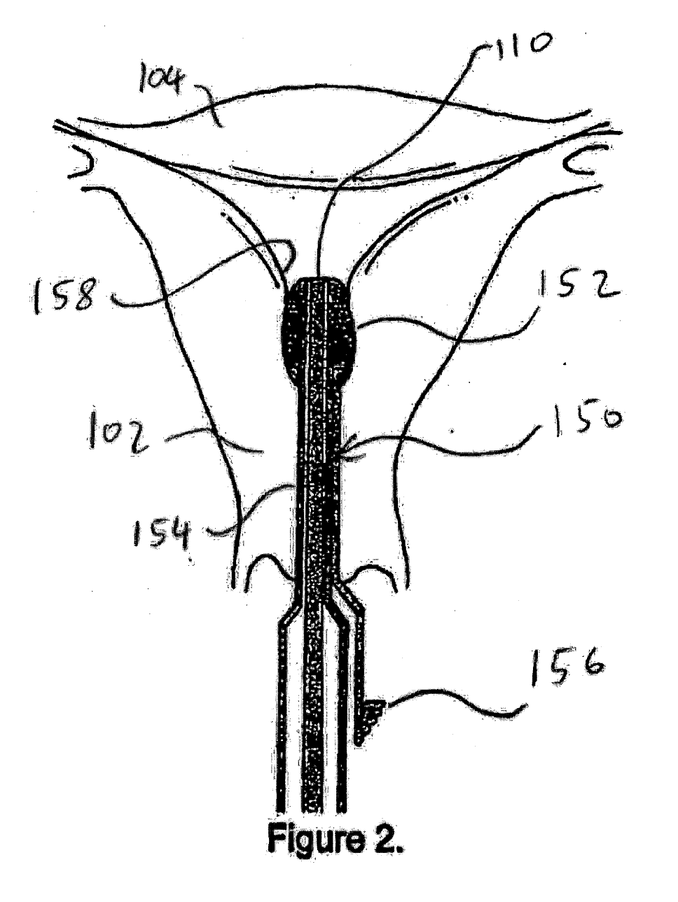

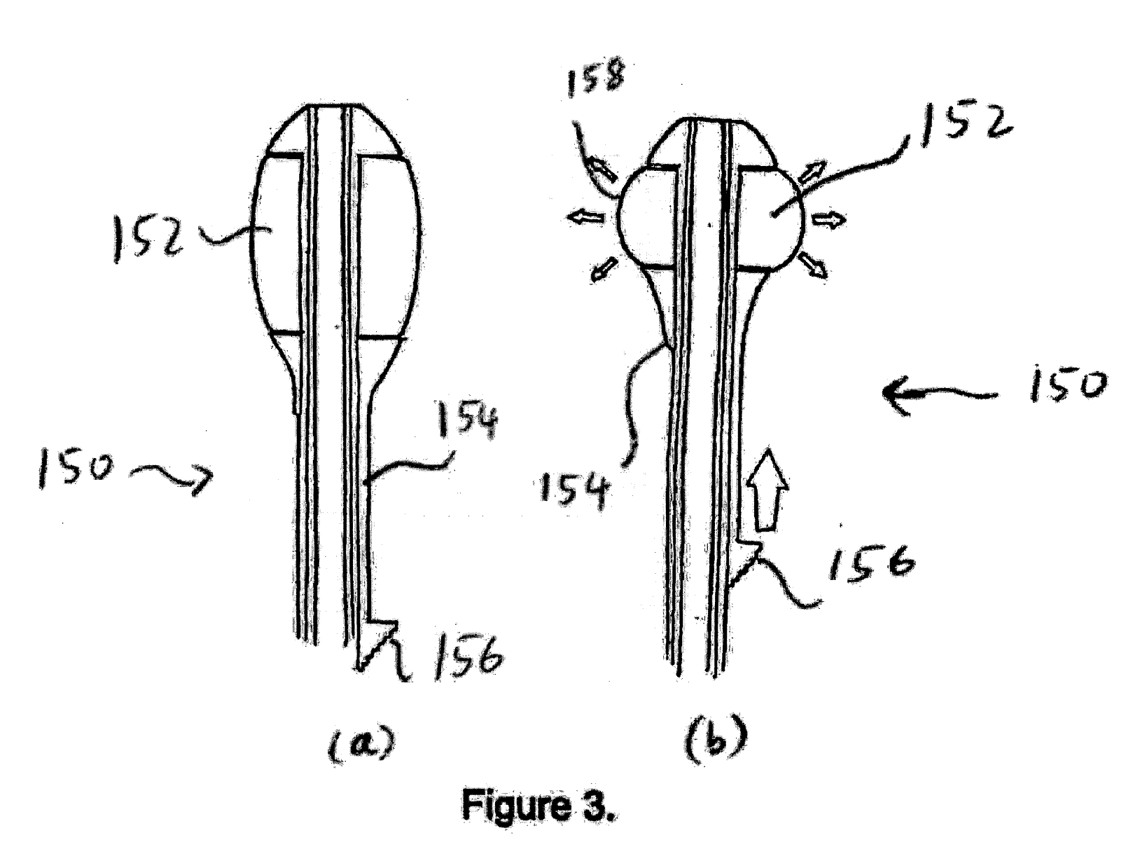

A further exemplary embodiment according to the present invention is shown in FIGS. 2 and 3. In this embodiment, the cervical seal 150 comprises a flexible element 152 designed so that, when compressed longitudinally, the flexible element 152 expands radially to increase the effectiveness of the seal against the cervix 102. For example, as shown in FIG. 2, the cervical seal 150 in configuration (a) has a flexible element 152 which is radially compressed so that a cross sectional area of the device is minimized to facilitate insertion through the cervix 102. In configuration (b), the flexible element 152 of the cervical seal 150 is compressed longitudinally, for example by moving a piston or rod 154 distally relative to the flexible element 152 which is held in place by a distal abutting surface 155 of an elongated member 157. In response to the longitudinal compression, the flexible element 152 expands radially to configuration (b), compressing the flexible element 152 against the cervix 102 and improving the seal thereagainst. A remote actuation member such as a tab 156 may be used to facilitate the operation of the rod 154 to control deployment of the flexible element 152.

The seal 150 is inserted, while in configuration (a) into a naturally occurring body orifice to the proximal opening of a hollow organ and, when located in a desired position, the tab 156 is pushed distally while maintaining the position of the elongated member 157 substantially constant, to expand the flexible member 152 against the wall of the organ sealing the opening thereto. For example, the seal 150 may be inserted into the cervix 102 in configuration (a) (FIG. 3), and placed so that the flexible element 152 is seated in the internal os, as shown in FIG. 2. Once the surgeon is satisfied with the placement of the seal 150, he deploys the flexible element 152 by displacing the remote tab 156, so that the rod 154 is translated distally relative to the elongated member 157. In this manner, the flexible element 152 is compressed longitudinally between the distal end of the rod 154 and the abutting surface 155 and expands radially to form a fluid seal within the cervix 102 effectively separating the uterus 104 from the vagina. Alternatively, the flexible element 152 may be located at another location along the cervical canal 102, in the center of the canal, for example, or closer to the vaginal opening of the cervix 102, depending on requirements of the medical procedure being performed.

Those skilled in the art will understand that the flexible element 152 is preferably formed of a material which exhibits significant radial expansion when compressed longitudinally. Alternatively, the flexible element 152 may be formed as a flexible membrane which bends to form a convex surface when longitudinally compressed. The material may also be sponge-like with at least portions being absorbent to help stop or absorb fluids with which it comes in contact. For example, a polymeric material may be used to form the flexible element 152 with a shape selected to mechanically facilitate its radial expansion. For example, the flexible element 152 may include a convex inner surface which, when longitudinally compressed by the rod 154, urges the outer surface of the flexible element 152 to bow outward to form a convex shape. The flexible element 152 may also include a sub-structure of mesh, ribs and/or reinforcements to further control the shape of the radially deployed flexible element 152, so that the outer surface 158 takes a preselected shape conforming to the shape of the internal os.

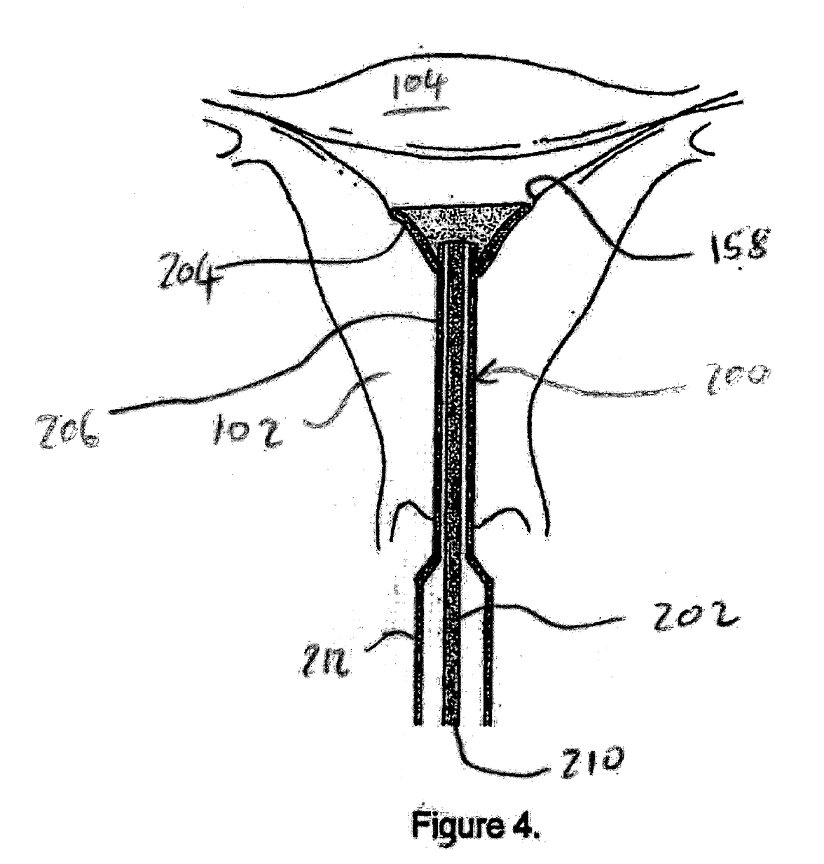

A seal 200 according to a further embodiment of the invention may also include a membrane which extends from the elongated body to close off the cervical opening. For example, as shown in FIG. 4, the seal 200 includes a membrane 204 disposed at the distal end of the elongated body 202. The membrane 204 is preferably formed of a flexible material, such as a polymer, so that it can conform to a shape of the opening of the cervix 102 to the uterus 104. According to this exemplary embodiment, the elongated body 202 is adapted to be inserted into the cervix 102 so that the distal end thereof and the membrane 204 are located within the uterus 104. More specifically, the membrane 204 is connected to an actuating element 206, which can be translated longitudinally relative to the elongated element 202. The actuating element 206 may form a sleeve around the elongated element 202, or may comprise one or more linkage bars extending through or along the elongated element 202. A hand operated control may be provided to permit the surgeon to translate the actuating element 206 when desired and a working channel 210 may be provided along the elongated element 202 to permit therapeutical instruments to be inserted into the uterus 104 through the cervical seal 200.

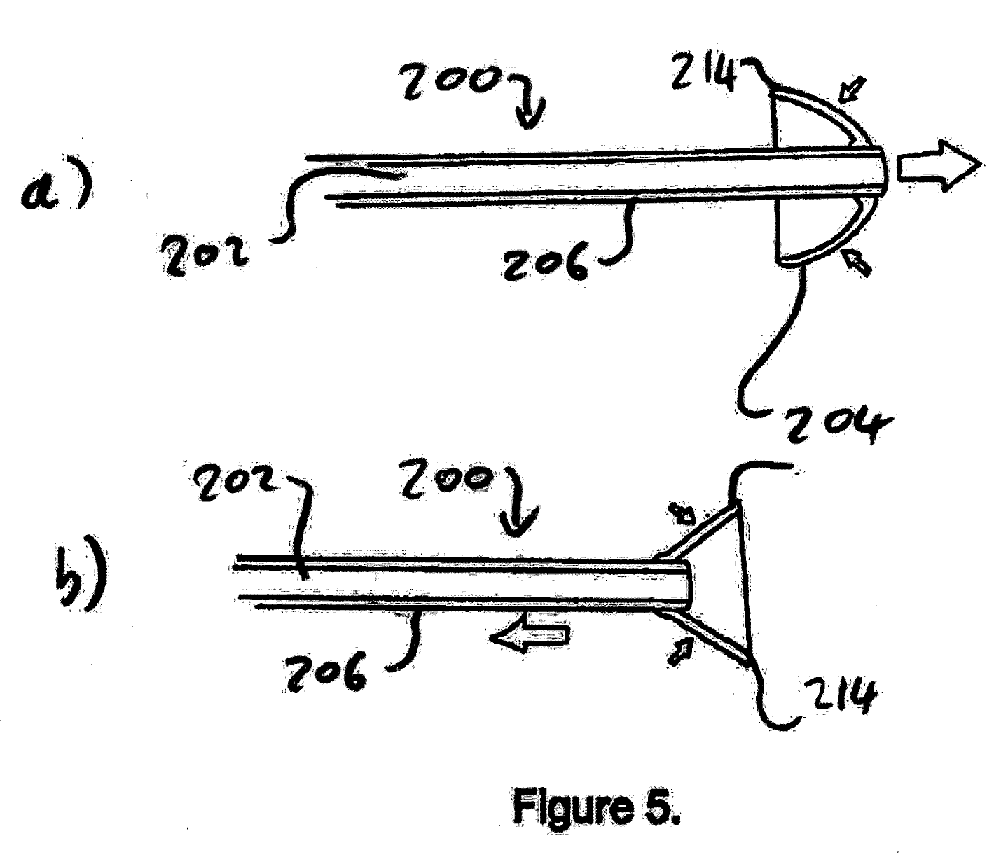

As shown in FIG. 5, the seal 200 may be inserted into the cervix 102 in an undeployed, insertion configuration (a) in which the membrane 204 is folded back toward the proximal end of the elongated element 202 forming a convex shape when viewed from the distal end. This configuration facilitates insertion of the seal 200 into the cervix 102 minimizing patient discomfort. In configuration (a), the actuating element 206 is positioned distally relative to the elongated member 202. After the cervical seal 200 has been positioned within the cervix 102 as desired, the actuating element 206 is translated proximally relative to the elongated shaft 202, for example by moving a hand operated control such as a slider 212 to invert the membrane 204 into a deployed, sealing configuration in which the membrane 204 forms a substantially conic shape opening toward the distal end, as shown in FIG. 5 (b). According to the embodiment shown, the membrane 204 forms an umbrella-like cuff which may be inverted to seal the cervical cavity, as shown in FIG. 5, by being pulled against the internal os of the uterus 104. Tension applied by the actuating element 206 may be used to invert the elastomeric cuff formed by the membrane 204 and to retain it in place against the distal opening of the cervix.

The shape of the deployed membrane 204 is preferably selected to match a shape of the internal os of the uterus 104. For example, the dimensions of the membrane 204 are preferably selected to match the deployed shape to the size and shape of the internal os of the uterus 104. Resilient elements such as rings 214, ribs and other structures may also be included in the membrane 204 to ensure that the deployed configuration sufficiently seals against the cervix 102.

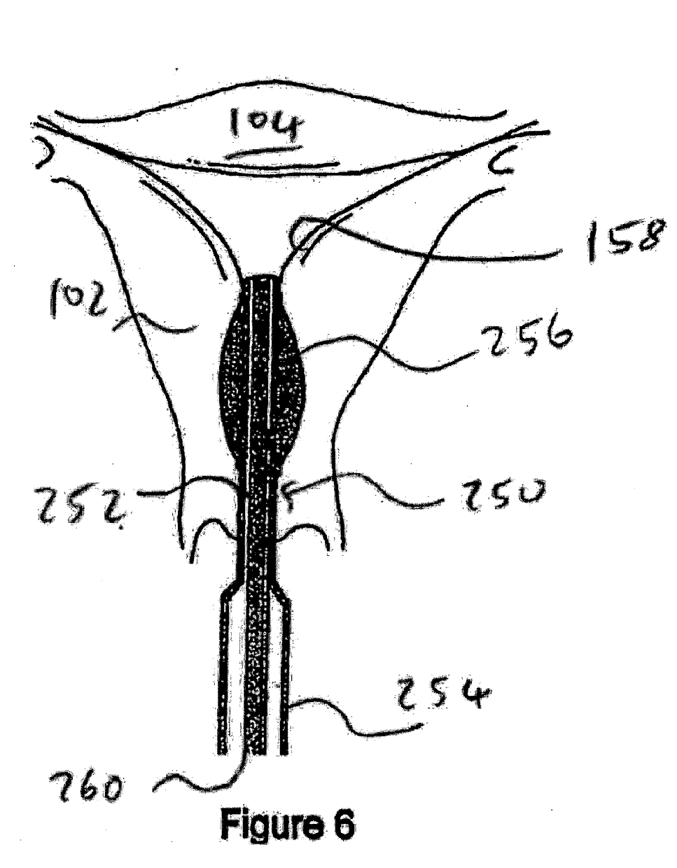

As shown in FIG. 6, a seal 250 according to a further embodiment of the present invention includes one or more inflatable elements which enhance the seal against the opening to a hollow organ. In this case, as before, the embodiment is described with reference to a cervical sealing device used to preform intrauterine medical procedures. As with the above described embodiments, the cervical seal 250 includes an elongated shaft 252 which has a distal end adapted to be inserted through the cervix 102 until a distal end thereof extends into the uterus 104. Medical devices (e.g., a histeroscope for irrigating the uterus with heated saline) may then be introduced into the uterus 104 through a working channel 260 of the elongated shaft 252.

A seal obtained between the elongated shaft 252 and the inner walls of the cervix 102 by expanding an inflatable element 256 which may, for example, be formed as a balloon. The inflatable element 256 is preferably placed near the distal tip of the seal 250, so that it is located near the inner os 158 of the uterus 104. It will be apparent to those of skill in the art, that the exact location of the inflatable element 256 along the length of the cervix 102 may be selected to achieve a desired level of resistance to the passage of fluids therepast. In one exemplary embodiment, the inflatable element 256 is connected to a source of inflation fluid via a conduit 254. The source of inflating fluid (not shown) may include, for example, a simple hand-syringe, a compressor, a pump, a pressure storage device, or any other means to provide a fluid under pressure to the conduit 254. A control for the supply of inflation fluid is preferably provided so that the operator of the device can inflate the inflatable element at a desired time (e.g., when the seal 250 has been properly positioned within the cervix 102. The inflating fluid is preferably selected to be non-toxic and biocompatible (e.g., compressed air or saline solution) so that a leak will not cause injury.

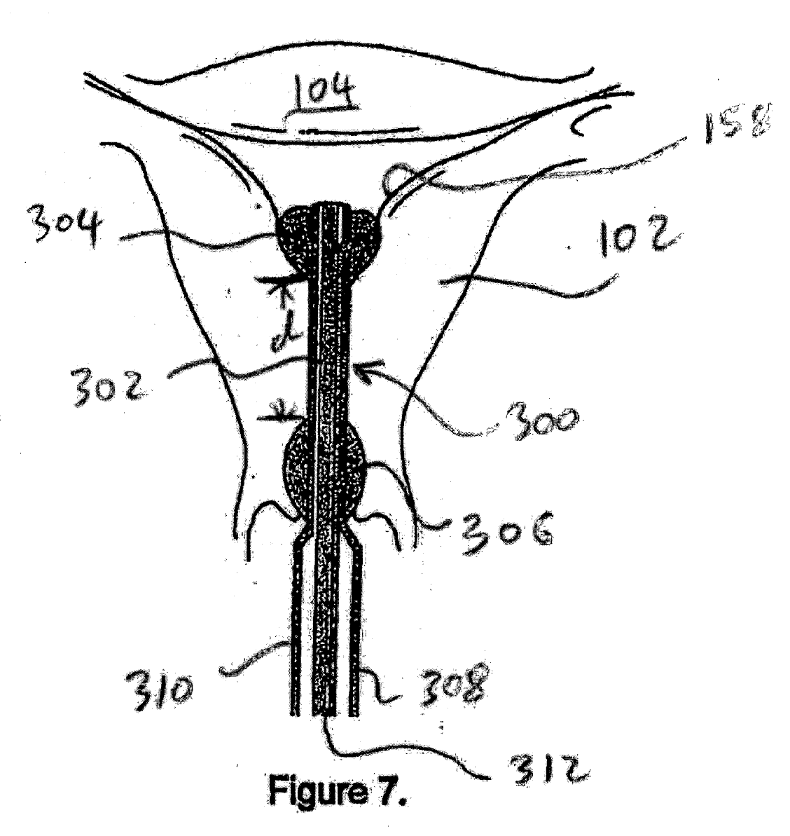

To further enhance the sealing of the cervix 102, additional inflatable elements may be employed. For example, as shown in FIG. 7, a seal 300 includes distal and proximal balloons 304, 306, respectively, are deployed from an elongated body 302, to form a double barrier to fluids flowing out of the uterus 104 through the cervix 102. In this exemplary embodiment, the inflatable elements 304, 306 may be inflated, for example independently from one another or at the same time, by supplying inflation fluid supplied through inflation conduits 308 and 310 which extend from the proximal end of the elongated body 302 to the inflatable elements 304, 306, respectively. As shown in FIG. 7, the distal balloon 304 is preferably deployed at the inner os 158 of the uterus 104, to form a plug at the distal opening of the cervix 102 while the second balloon 306 is deployed more proximally (e.g., near a proximal end of the cervix 102. For example, a distance “d” between the balloons 304, 306, is preferably selected so that contraction of the cervical muscles closes the walls of the cervix 102 around the elongated body 302 between the two balloons.

In the exemplary embodiments shown in FIGS. 6 and 7, the seals 250 and 300 are inserted into the cervix 102 with the inflatable elements deflated, in an insertion configuration. After the cervical seals 250, 300 have been correctly positioned, inflating fluid is supplied and the inflatable elements 256, 304 and 306 are inflated to seal the cervix 102. An inflatable element may also be deployed at the distal end of the cervix 102, inside of the uterus 104, to anchor to the seal 250, 300 therein. The amount of inflation fluid supplied to provide may be varied depending on the size of the cervix 102 and on the type of procedure to be carried out.

When there is no longer a need for the barrier provided by the seal 250, 300, the inflatable elements are retracted by deflating them and withdrawing the seal 250, 300 from the body. For example, after an ablation procedure has been completed and the ablating fluid has been drained from the uterus, the inflating fluid may be removed from the inflatable elements 256, 304 and 306, so that they will collapse to the insertion configuration. The seal 250, 300 may then be removed from the body.

As described above, an exemplary seal according to the present invention may be inserted trans-cervically so that distal end thereof is located within the uterus 104. A seal is then established preventing the migration of fluids from the uterus 104 through the cervix 102 (e.g., by deploying sealing elements therefrom) and medical devices are introduced into the uterus 104 through the working channel of the seal to carry out a medical procedure (e.g., endometrial ablation) while preventing fluids, such as heated saline solution, introduced into the uterus for the procedure from escaping through the cervix 102 and damaging non-targeted tissue. After the procedure has been completed, the seal is withdrawn (e.g., after retracting sealing elements thereof).

The present invention was described with reference to specific exemplary embodiments. Those skilled in the art will understand that changes may be made in details, particularly in matters of shape, size, material and arrangement of parts. Accordingly, various modifications and changes may be made to the embodiments. For example, the exemplary devices described may be used to provide a fluid seal to openings of bodily cavities or hollow organs other than the cervix. The specifications and drawings are, therefore, to be regarded in an illustrative rather than a restrictive sense.

What is claimed is:

1. A device for accessing a hollow organ comprising:
an elongated body sized for insertion into an opening into a hollow organ, the elongated body including a working channel extending therethrough from a proximal opening which, when the seal is in an operative position, is located proximally of the opening to a distal opening which, when the seal is in the operative position, opens into the hollow organ; and
a seal extending around a portion of the elongated body between the proximal and distal openings, the seal expanding from an insertion configuration to a sealing configuration.

2. The device according to claim 1, wherein the seal comprises a flexible element coupled to an actuation member, wherein actuation of the actuation member moves the flexible element between the insertion and sealing configurations.

3. The device according to claim 2, wherein the actuation member is a rod movable along an axis substantially parallel to the axis of the elongated body, actuation of the rod longitudinally compressing the flexible member to radially expand the seal to the sealing configuration.

4. The device according to claim 2, wherein the seal extends from a distal end of the elongated body so that, when in the insertion configuration, the flexible member extends proximally from the distal end to form a convex shape as viewed from the distal end and, when in the sealing configuration, the flexible member extends distally to form a convex shape as viewed from the distal end.

5. The device according to claim 2, further comprising an actuator coupled to a proximal end of the actuation member wherein, when in the operative position, the actuator extends proximally out of the opening.

6. The device according to claim 4, wherein, when in the sealing configuration, the flexible member engages a distal surface of the opening.

7. The device according to claim 6, wherein the flexible element comprises a resilient element biasing the flexible element toward a desired shape when in the insertion and sealing configurations.

8. The device according to claim 7, wherein actuation of the actuating element when in the insertion configuration applies a tension to the flexible member to pull it proximally against a distal surface of the opening.

9. The device according to claim 1, wherein the seal is formed of an absorbent material so that, when inserted into the hollow organ, fluids are absorbed thereby expanding the seal to the sealing configuration.

10. The device according to claim 1, wherein the seal includes a first inflatable element.

11. The device according to claim 10, wherein the first inflatable element comprises a balloon located at a distal end of the elongated body.

12. The device according to claim 10, further comprising a second inflatable element located proximally of the first inflatable element.

13. The device according to claim 10, further comprising an inflation lumen extending from the first inflatable element to a proximally opening for receiving an inflation fluid therein.

14. The device according to claim 9, wherein the absorbent material is a high expansion foam.

15. The device according to claim 15, wherein the seal further comprises an expanding weave portion.

16. The device according to claim 15, further comprising a removable protective sheath to cover the high expansion foam element during insertion into the hollow organ.

17. The device according to claim 15, wherein the high expansion foam element extends longitudinally along a length of the elongated body which, when in the operative position, extends between proximal and distal ends of the opening.

18. The device according to claim 11, wherein, when in the operative position, the first inflatable element abuts a distal surface of the opening to anchor the elongated body in the operative position.

19. A sealing device, comprising:
an elongated member insertable into a hollow organ through a naturally occurring body opening, the elongated member defining a working passage extending longitudinally therethrough; and
a sealing element extending around a portion of the elongated member which, when the elongated member is in an operative position within the hollow organ, is disposed between distal and proximal ends of the opening, the sealing element expanding from an insertion configuration to a sealing configuration.

20. The sealing device according to claim 19, wherein the sealing element further comprises an umbrella-shaped membrane defining a reversible cuff placeable over a distal surface of the opening.

21. The sealing device according to claim 20, further comprising an actuator coupled to the membrane to move the cuff between the insertion and sealing configurations.

22. The sealing device according to claim 20, wherein the membrane further comprises a resilient element biasing the membrane to desired shapes in both the insertion and sealing configurations.

23. The sealing device according to claim 19, further comprising an actuator coupled to the sealing element, wherein the sealing element includes a flexible element coupled to the actuator so that, when compressed longitudinally by the actuator, the flexible element expands radially to the sealing configuration when compressed longitudinally.

24. The sealing device according to claim 19, wherein the sealing element includes a first inflatable member.

25. The sealing device according to claim 24, further comprising an inflation conduit coupled to the first inflatable member to provide inflating fluid thereto.

26. The sealing device according to claim 24, wherein the inflatable element is disposed near a distal tip of the elongated portion so that, when in an operative position, the inflatable element is located within a distal end of the opening.

27. The sealing device according to claim 24, wherein the sealing element further comprises a second inflatable element mounted around the elongated portion proximal to the first inflatable element so that, when in an operative position, the second inflatable element is located between proximal and distal ends of the opening.

28. The sealing device according to claim 19, wherein the sealing element comprises a high expansion foam element disposed around a distal portion of the elongated body, a thickness of the high expansion foam element increasing as fluids are absorbed thereinto.

29. The sealing device according to claim 28, further comprising a protective sheath which, when in the insertion configuration, surrounds the high expansion foam element, the sheath being slidably removable therefrom to expose the foam element when the device is in the operative position.

30. The sealing device according to claim 28, wherein the foam element comprises a portion formed of expanding weave material.
